(19) United States
(12) Patent Application Publication
(10) Pub. No.: US 2005/0042308 A1
(43) Pub. Date: Feb. 24, 2005

(54) Pharmaceutical composition having cholagogic and litholytic effect and its preparation method

(76) Inventors: Wei Xiao, Jiangsu (CN); Yin Yang, Jiangsu (CN); Ya Ling, Jiangsu (CN); Minghui Li, Jiangsu (CN); Zhenggen Liao, Jiangsu (CN); Chenfeng Zhang, Jiangsu (CN); Shubo Cao, Jiangsu (CN)

Correspondence Address: EDWARDS & ANGELL, LLP, P.O. BOX 55874, BOSTON, MA, 02205, US

(21) Appl. No.: 10/494,143

(22) PCT Filed: Sep. 5, 2002
(86) PCT No.: PCT/CN02/00622

(30) Foreign Application Priority Data
Oct. 29, 2001 (CN) 01134090.8

(51) Int. Cl. A61K 35/78

(52) U.S. Cl. 424/747; 424/766

(57) ABSTRACT

The invention publishes a kind of drug complex with cholagogue and lithontriptic effect and its preparation method; the invention also publishes a kind of quality-control method of a kind of drug complex soft capsule. The invention drug complex is composed by the main materials of mint oil and herit oil. The aforementioned drug is mixed uniformly according to certain proportion, and can be added into conventional accessories to prepare into clinically accepted forms of drug, such as pill, tablet, orally-taken liquid preparation, capsule, granule et al. To control the drug quality according to the quality-control method of the soft-capsule-form drug publicized in the invention, good product stability and high repeatability can be acquired; The invention drug complex has distinct cholagogue, lithontriptic, anti-inflammation, analgesic effect through the pharmacological experiment.

### TECHNICAL FIELD

The invention refers to a drug complex and its preparation method, especially refers to a drug complex with cholagogue and lithontriptic effect and its preparation.

### BACKGROUND TECHNOLOGY

The cholelithiasis is a kind of common disease with high incidence harming the health of people, which is one cause of the common colica syndrome inland and abroad. In the past, surgical operation is the main therapeutics for the cholelithiasis. But this therapeutics not only makes patients suffer from pain of operation, but also make the postoperative residual gallstone incidence higher. Thereinto, the incidence of residual gallstone in the hepatic bile duct is even increased to 70-92%, so the second surgical operation is required, which is more difficult. In the ending of 1960s and the beginning of 1970s, cholanic acid lithontriptic drug was applied in the treatment of the cholelithiasis in abroad and was somewhat effective. Now the drug is used inland too. According to the clinical case report, the efficacy of the therapeutics is about 50% (including reducing the gallstone size, dissolving the gallstone or decreasing the gallstone effect). According to the lithontriptic efficacy, it is significantly effective to the gallstone with diameter less than 1.0 cm. However, it is less effective or even not effective to the gallstone with diameter more than 2.0 cm, and has no obvious effect in the long-term treatment of 6 months for some of these gallstones. The lithontriptic speed of cholanic acid is slow, and in general 6˜24 months are required. The therapeutics for the treatment of the cholelithiasis can make 20% of the patients' SGOT increase. So it has certain toxicity. Many inland researches and experiments have been done for the lithontriptic treatment of the gallstone. Applying gallstone-excreting drug made from Chinese herb medicine to excrete the gallstone for the treatment of the cholelithiasis is comparatively effective. The efficacy for excreting the gallstone in the hepatic bile duct is 50˜80%, but the therapeutics is less effective or even not effective for excreting big gallstone.

### TECHNIQUE CONTENT

The aim of the invention is to provide a drug complex with cholagogue and lithontriptic effect and its preparation; the purpose of the invention is also to provide a quality control method for the soft-capsule-form drug.

The purpose of the invention is achieved through the following technique project:

Mint oil: Herit oil 2-6:1-2 volume ratio, the ratio of the invention drug complex is also Mint oil: Herit oil 3:1 volume ratio, Mint oil: Herit oil 4:1 volume ratio.

The aforementioned drug is mixed with certain volume ratio into which the conventional accessories is added, and then is prepared into the clinically acceptable dose type, e.g. pill, tablet, oral-liquid-form drug, capsule, granule-form drug etc.

The quality control method for soft-capsule-form drug dose of the invention:

Identification: Get 1 drop of soft capsule content, add 3-5 drops of H₂SO₄ and a little amount of vanillin crystal, it should be orangered, then add 1 drop of water, it should be violet;

Get 1 ml of soft capsule content, add 10 ml acetoacetate, and then mix them as sample solution for test; additionally get mint added with acetoacetate to prepare the control mint solution with 1 mg/ml concentration. According to the thin-layer chromatography experiment, aspirate 2 ul from each of the aforementioned three kind of solution respectively, drop them onto the same silica-gel G thin-layer plate, use 12-18:2-4 petroleum ether-acetoacetate at 30-60° C. as developer, develop them below 25° C., then get them out and drying them, spray with newly prepared 5% vanillin sulphate solution, blow with hot wind until the dot is clear. The chromatogram of the test solution has the same color dot in the corresponding position with the control solution chromatogram;

Relative density: get soft capsule content, measure the relative density according to the relative density assay method, the relative density should be 0.881˜0.901;

Refractive index: get soft capsule content, measure refractive index legally (Appendix VII F, Volume I, Chinese Pharmacopoeia, edition in 2000), the refractive index should be 1.456˜1.466;

Optical rotatory power: get content as the relative density measurement, measure Optical rotatory power legally (Appendix VII E, Volume I, Chinese Pharmacopoeia, edition in 2000), the specific rotation should be −14°˜22°;

Content Measurement:

1. The Measurement of Ester Content and Total Alcohol Content Get soft capsule content about 5 g, quantify minutely, place them into the flask, add 10 ml ethanol which is indicated neutral by phenolphthalein indicator solution, mix them, and add 2 drops of phenolphthalein indicator solution. At first, neutralize the free acid with KOH solved in 0.1 mol/L ethanol, then add minutely 25 ml 0.5 mol/L KOH volumetric solution solved in ethanol, reflux for 0.5-1.5 hours in water bath, cool them, then add 0.5 ml phenolphthalein indicator solution, use 0.5 mol/HCL to slowly titrate the residual KOH, make the blank test at the same time. 1 ml 0.5 mol/L KOH volumetric solution correspond to 99.15 mg menthol acetate (C₁₂H₂₂O₂); through the corresponding mass of the menthol acetate, the ester content of each soft capsule content should be 5-11% (g/g);

Acetylation: get 10 ml soft capsule content, place them into acetylating flask with 100 ml volume, add 10 ml acetic acid and 2 g newly solved anhydrous sodium acetate, with air condenser attached to the flask, placed into 145±3° C. oil bath or sand bath, boiled for 0.5-1.5 hours, get them out and cool, through condenser add 50 ml, place into water bath, vibrate, heat for 10-20 minutes, then cool them; placed into dispenser funnel, get rid of the underlayer acidic solution; add 50 ml saturated NaCl solution, vibrate fully, stationary fractionation, get rid of upper layer water solution, then wash with water for several times, 40-60 ml every time, until the washing solution is indicated neutral by phenolphthalein indicator solution; place the acquired acetylating oil into 25 ml Erlenmeyer flask with plug, add 3 g anhydrous sodium sulphate, close the flask tightly with the plug, and vibrate ever and agah, make dehydration until when getting 1 drop of acetylating oil mixed with 10 drops of CS₂ no turbidity occurs, then filtrate with dry filter paper;

Saponification: get about 1 g dry acetylating oil, quantify minutely, place into 100 ml Erlenmeyer flask, add 25 ml KOH solution solved in 0.5 mol/L ethanol, reflux for 0.5-1.5 hours in water bath, cool, take the condenser away, drop 0.5 ml phenolphthalein indicator, use 0.5 mol/L HCL solution to titrate the residual KOH, make the blank test at the same time. Calculate as the following formula, we can get the result:

$$Total alcohol content \% = \frac{( B - A ) \times N \times 0.1543}{W - ( B - A ) \times N \times 0.04204} \times 100 \%$$

B: the volume of 0.5 mol/L HCl standard solution consumed in the blank test (ml);

A: the volume of 0.5 mol/L HCL standard solution consumed by acetylating oil (ml);

N: molar concentration of standard solution;

W: mass of the acetylating oil (g);

0.1543: the milligram molar number of geraniol;

0.04202: the milligram molar number difference between acetate and ketol;

Through the corresponding mass of geraniol, the total alcohol content of each soft capsule should not be less than 50.0%;

2. The Measurement of Menthol and Geraniol Content

Referring to the gas chromatography test method (page 40, Appendix VI E, Volume I, Chinese Pharmacopoeia, edition in 2000).

Measure the methanol and geraniol content through gas chromatography test method, chromatography condition and system adaptability experiment capillary chromatographic column: it is best to use 5% diphenyl-95% dimethyl silicane copolymer as solid capillary chromatographic column; sample inlet temperature: 200-250° C.; detector temperature: 200-250° C.; make sample measurement with the distribution ratio of 25-100:1; heat-up progressively, initial temperature 70-90° C., maintain for 2-3minutes, heat-up to 150-180° C. with the heat-up speed 4-15° C. every minute, maintain for 24 minutes; flow rate of carrier gas: 1-2 ml/min; According to the geraniol peak theoretical plate number should not be less than 300000;

Correction factor measurement get moderate n-pentadecane or n-tridecane or n-tetradecane or naphtalene or camphor or n-undecane etal as internal standard control sample, measure precisely, add acetoacetate to prepare into 2.5 mg/ml concentration solution as internal standard solution; Get adequate amount of menthol geraniol control sample, measure precisely, add acetoacetate to prepare into solution with menthol 6 mg/ml concentration, geraniol 1 mg/ml concentration, aspirate precisely aforementioned three kinds of solution 2 ml respectively, placed into 10 ml measuring flask, add acetoacetate, dilute it to the scale, vibrate uniformly, aspirate 1 ul, injected into gas chromatograph, measure, measure the correction factor;

Get about 50 mg soft capsule content of loading diversity item, measure precisely, placed into 10 ml quantifying flask, add 2 ml internal standard solution minutely, add acetoacetate, dilute it to the scale, vibrate uniformly, aspirate 1 ul, injected into gas chromatograph, measure, then we can acquire: according to the menthol(C₁₀H₂₀O), the mint oil in each soft capsule content should not be less than 79 mg; according to the geraniol(C₁₀H₁₈O), the Herit oil should not be less than 7 mg;

The pharmaceutical effect experiment results of the invention drug complex shows: forcing to take the invention drug complex capsule (Cholagogue and lithontriptic soft capsule) stock solution (abbrev. LDRS) 0.077 ml, 0.154 ml, 0.307 ml/Kg.d, respectively relative to 3.85, 7.7, 15.4 times of clinical dose for each body weight kilogram, forcing to take medicine one time each day, last for 90 days, make the lithogenic rate drop respectively to 45.4%, 27.3%, 0.0% respectively from 91%, have significant lithogenesis inhibition effect, and decrease the serum glycocholic acid, total bilirubin and free bilirubin of guinea pig and normal mouse, decrease the total gallbladder weight of the lithogenic guinea pig, present good lithogenisis prevention effect; Different concentration LDRS have different lithontriptic effect to two kind of human gallstone, especially have good lithontriptic effect and speed for the bilirubin gallstone and mixed type gallstone. In general, 24-48 hours later after adding the drug solution, there is red-yellowish color in the lithontriptic drug solution. Lithontriptic experiment in vivo shows: different concentration LDRS have significant lithontriptic effect for the gallstone of the rabbit, different dose cholagogue and lithontriptic soft capsule can improve bile secretion coefficient of the rat. Comparing to the cholagogue Cholan-DH, it has faster effect, larger bile secretion coefficient, and can maintain longer effect, and has distinct cholagogue effect and can decrease the bilirubin content of bile. Using three kinds of dose LDRS to treat the mouse infected by Staphylococcus aureus, experiment shows LDRS have good prophylactic effect for Staphylococcus aureus infection. Exsomatized guinea pig intestine canal gallbladder muscle stripe experiment shows c7holagogue and lithontriptic soft capsule has significant inhibition effect to the exsomatized smooth muscle(intestine/gallbladder), and can antagonize the muscle stripe spasm caused by acetylcholine, histamine, BaCl and has good spasmolytic effect.

Through forcing to take the aforementioned three kinds of dose Cholagogue and lithontriptic soft capsule, the content of soft capsule has different degree anti-inflammation effect, not only for the acute inflammation, e.g.the auricular edema of the mouse caused by dimethylbenzene or the foot swollen of the rat caused by the carragheen, but also for the granulation proliferation inflammation caused by agar. Forcing to take Cholagogue and lithontriptic soft capsule has good antipyretic effect for the fever of the rat caused by 2, 4-dinitrophenol; and significantly decrease the capillary permeability caused by dimethylbenzene. Forcing to take different dose Cholagogue and lithontriptic soft capsule has certain inhibition effect for the body-twisting reaction caused by acetic acid and foot-licking reaction, and has certain analgesia effect.

Experiment sample 1: Lithogenesis prophylaxis effect for the animal experimental gallstone of the Cholagogue and lithontriptic soft capsule.

1. Experimental Materials:

1.1 Testing drugs: There is 0.4 ml oil-like substance in each Cholagogue and lithontriptic soft capsule (named stock solution in the below). The unencapsulated stock solution is used in this experiment, which is light green oil solution. The batch number of Cholagogue and lithontriptic soft capsule (named “LDRS” in the below) is 970101. For the purpose of diluting stock solution easily, add them into 1% Tween-80 to emulsify. After drug emulsification, stored for the future use in the refrigerator. When using, dilute to the needed concentration with 0.5% carboxymethyl cellulose as diluter. LDRS stock solution is provided by KangYuan Pharmaceutical Co., Ltd. in Lianyungang city. Three kinds of highs, moderate and low dose are used in the experiment, which is respectively 0.077 ml/kg.d, 0.154 ml/kg.d, 0.307 ml/kg.d, respectively relative to 3.85, 7.7, 15.4 times clinical dose every kilogram body weight. The following experiments are all proceeded with the aforementioned three kinds of dose.

Positive-control drug: Eulektrol (which is imported capsule for gallstone treatment, with similar appearance to LDRS, 0.2 ml oil solution in each capsule, 100 mg effective component in each capsule). The content of soft capsule is produced by PHARMACEUTICAL FACTORY (HAMBURG GERMANY). Chinese Importation Certification Number 880209REGHK-22425, drug batch number 1291 chB11742EXP1295, The drug preparation: add its oil solution into 1% Tween-80 to proceeding emulsification, to prepare into 0.015 ml/ml with 0.5% carboxymethyl cellulose. The dose for each kilogram body weight of guinea pig is 0.15 ml/Kg.d, 15 times of the clinical dose for each kilogram body weight.

1.2 Main Experimental Drugs

Feeding stuff with promoting gallstone formation effect: Model building drugs composed of fundamental feeding stuff and feeding stuff with promoting hyperlipaemia effect.

Fundamental feeding staff: composed of corn powder 50%, wheat husk 10%, minced rice powder 10%, fish powder 6%, bean whiting 17%, CaCO₃ 1%, salt 1%, yeast powder 1%, wheat powder or corn powder 4%, which is provided by Sichuan Institute of Chinese Materia Medica.

Feeding stuff with promoting hyperlipaemia effect: composed of 1% casesin(produced by The microbial culture medium manufacturing Factory of Haidian district in Beijing) , 1% cellulose(produced by chemical factory of LuZhou city in Sichuan province, batch number 970101), 0.02% cholalic acid (purchased from the Chemical reagent shop of ChongQing city), 0.5% Cholesterol(Product of Merck Pharmaceuticals Co., Ltd. purchased from the Chemical reagent shop of ChongQing city) 1.5% sucrose, 1% pig meat oil(purchased from the local market).

¹²⁵I radioimmunoassay kit: produced by Chinese Institute of Atomic energy, batch number IMK-407, Certification Document Number (94) Hygiene Drug Word R-13.

1.3 Main Testing Instrument:

semi-automated biochemical analyzing machine, made by France Secomam Co., Ltd, model S-500P; Immunoassay machine, made by 262 Factory of XiAn city of china; Photoelectronic balance, model BP3100, with TOP03 model multifunctional automated printer, the measurement scale is 600, 1200, 3200 g: sensitive weight is 0.01, 0.02, 0.05 g respectively, made by German Sartorius electronic Co., Ltd; photoelectronic balance, TP1000, measurement scale is 1000 g, sensitive weight is 0.1 g, made by HuNan XiangYi weighting apparatus Co., Ltd, batch number 961183; LDs-2A centrifuge, made by BeiJing medical centrifuge Factory, bath number 970609; GALEN- III binocular biological Medical centrifuge Factory, made by ShangHai JiangNan microscopic factory, exportation type product, with automated light source, the magnifying number can reach to 1600; JN-A precise torsion balance: measurement scale 500 mg, sensitive weight 1 mg, made by the ShangHai No.2 Balance Factory.

1.4 Animal guinea pig: heathly guinea pigs with 250-300 g body weight are used in the experiment, provided by the experimental animal center of the preclinical medical college of HuaXi Medical University.

Mouse: distant line of descen grade I qualified KunMing species mouse (closed for more than 30 generations), qualification certification registration number: No.85 experimental animal quality-control (95).

Rat: Distant line of descen grade I qualified SD species rat (closed for more than 30 generations), qualification certification registration number: No.92 experimental animal quality-control (95). Rat and mouse are all provided by the experimental animal center of Sichuan antibiotics industry institute.

Japanese white rabbit with big ear: body weight 2-2.5 kg, both female and male white rabbit, Qualification Certification Registration Number: No.24361040 Experimental animal Registration Number of the First medical university, local registration number: No. 40 experimental animal quality-control (95), provided by Sichuan Institute of Chinese Materia Medica.

1.5 Experimental Environments

The experiment is proceeded in the pharmacological base for new drug in Chinese medicine and Chinese herb administration bureau of Sichuan province. The room temperature Of the lab is controlled at 18˜28° C., relative humidity 40˜60%, combining fluorescent and sunlight illumination, 12 hours bright environment and 12 hours dark environment, equipped with automated gas exchange equipment. The experimental animals are fed in separate cages, not more than 5 animals in each cage, feed routinely with soft feeding stuff (feeding stuff with whole potential values), 20 g soft feeding stuff/100 g.bw for each rat everyday, 6 g soft feeding stuff/10 g.bw for each mouse everyday, and moderately feed with green feeding stuff.

1.6 Data Analysis

Quantitative data is expressed with x±SD; use T-test to analyze the results (between experimental group and control group, or between the group before drug-administration and the group after drug-administration).

Enumerative data is analyzed using x²test between the experimental group and control group.

2. The Lithogenesis Prophylaxis Effect for Bilirubin Gallstone in Guinea Pig.

Get 61 motley healthy female guinea pigs with 250˜300 g body weight. 1 week environment adaptation later after purchasing, the guinea pigs are separated into 6 groups randomly; Thereinto 5 groups are experimental groups. There are 11 guinea pigs in the first group, which is promoting gallstone formation model group [named gallstone formation group in the following, every guinea pig in this group is fed with 20 g/100 gbw (bw is abbreviation for body weight) feeding stuff with promoting gallstone formation effect, the following groups are all fed according to the method]. There are 11 guinea pigs in the 2nd, 3rd, 4th group, forcing to take LDRS stock solution 0.077 ml/kg.d, 0.154 ml/kg.d, 0.307 ml/kg.d, and feeding stuff with promoting gallstone formation effect. The 11 guinea pigs in the 5th group are fed with Eulektrol 0.1 5 ml/kg.d and feeding stuff with promoting gallstone formation effect. The guinea pigs in the aforementioned 5 groups are forced to take control drug and test drug at 8˜9 O'clock AM, in the afternoon measure every guinea pig respectively and feed them with feeding stuff with promoting gallstone formation effect, the rest 6 guinea pigs are all fed with fundamental feeding stuff. To ensure the accuracy of forcing to take drugs to experimental mouse/rat, single cascade in the feeding cage is used in the experiment; half cascade in the feeding cage is used for feeding guinea pig. The animals in every group are trained to be fed in daytime after grouping. At the same time, when feeding, uniformly mix the food and drug repeatedly. The food-adding inlet of crib can only accommodate the mouth of guinea pig, and then the food is added from the top down. Feeding time is separated into two phases, at the first phase the animals are fed with feeding stuff mixed with drug (or feeding stuff with promoting gallstone formation effect), when the feeding stuff mixed with drug is completely consumed the animal are fed with the fundamental feeding stuff. The animals in the experiment are fed with drug and feeding stuff with promoting gallstone formation effect for 90 days, sever the femoral vein of the animals in every group after 24 hours at the last drug administration time (isolate the serum), then execute the animals through decollation, rapidly anatomize the thorax and abdomen, clamp the common bile duct with hemostat, get the gallbladder to examine carefully and record the number of lithogenesis mouse/rat, calculate the rate of lithogenesis (the number of lithogenesis mouse/the number of experimental mouse), the result is showed in Table 1. According to the manipulation procedure of the ¹²⁵I glycocholic acid radioimmunoassay kit, the glycocholic acid content of serum is measured respectively using γ immunoassay machine. Using the bilirubin assay kit made by the RONGSHENG reagent factory of biologic researching institute of Chinese Science Academy, through the caffeine method, to measure the common bilirubin and conjugated bilirubin respectively, calculate the free bilirubin, the result is showed in Table 1 and Table 2.

From the Table 1, continuously forcing to take LDRS stock solution 0.307 ml/kg.d , 0.154 ml/kg.d , 0.077 ml/kg.d for 90 days has different inhibition effect for the bilirubin gallstone formation rate of the guinea pig caused by the feeding stuff with promoting gallstone formation effect, thereinto the inhibition effect to the bllirubin gallstone formation rate of 0.307 ml/kg.d and 0.154 ml/kg.d dose are 0.0%, 27.3%, comparing to the lithogenesis group fed with the feeding stuff with promoting gallstone formation effect, the inhibition rate are 90.9% and 63.6% respectively. Comparing to the lithogenesis rate of the group fed with the feeding stuff with promoting gallstone formation effect, it is significantly lower, P<0.01 or 0.001,has significant difference showing that continuously forcing to take LDRS has good lithogenesis prophylaxis effect for bilirubin gallstone in guinea pig caused by feeding stuff with promoting gallstone formation effect.

TABLE 1
the effect of continuously ig different dose LDRS stock solution
90 days to lithogenesis rate and gallbladder weight

| | | | lithogenesis rate | | |
|---|---|---|---|---|---|
| | | | (lithogenesis animal | inhibition | |
| | animals | dose | number/experimental | rate | dry gallbladder |
| Group | number | (ml/kg) | animal number) | (%) | weight (mg · x ± SD) |
| Eulektrol | 11 | 0.150 | 8/11 | 18.2 | 26.60 ± 6.3** |
| LDRS high dose | 11 | 0.307 | 0/11** | 90.9 | 19.36 ± 6.2***^{ΔΔ} |
| LDRS moderate dose | 11 | 0.154 | 3/11** | 63.6 | 23.80 ± 5.7*** |
| LDRS low dose | 11 | 0.077 | 5/11 | 45.4 | 22.80 ± 4.8*** |
| Lithogenesis | 11 | 0.5% CMC | 10/11 | — | 39.50 ± 10.2 |
| normal control | 6 | — | 0/6 | — | 20.70 ± 1.90***^{ΔΔ} |

Compared with lithogenesis group
*P < 0.05,
**P < 0.01,
***P < 0.001
The inhibition rate = gallstone formation rate of control group − gallstone formation rate of experimental group
Compared with Eulektrol
^{ΔΔ}P < 0.01

TABLE 2
the influence on the serum glycocholic acid and
bilirubin level of LDRS and Eulektrol

| | guinea pig | dose | glycocholic acid | serum bilirubin content | | |
|---|---|---|---|---|---|---|
| Group | number | (ml/kg) | (μg/dl · x ± SD) | T-BIL | D-BIL | F-BIL |
| Eulektrol | 8 | 0.150 | 535.47 ± 167.4 | 1.80 ± 0.35 | 0.53 ± 0.10 | 1.27 ± 0.24 |
| LDRS high dose | 7 | 0.077 | 411.27 ± 129.3* | 1.68 ± 0.22** | 0.42 ± 0.05** | 1.26 ± 0.17 |
| LDRS moderate | 8 | 0.154 | 406.76 ± 105.9** | 1.63 ± 0.22** | 0.47 ± 0.06** | 1.16 ± 0.16** |
| dose | | | | | | |
| LDRS low dose | 10 | 0.307 | 386.70 ± 47.4*** | 1.58 ± 0.21** | 0.54 ± 0.07** | 1.05 ± 0.14*** |
| Lithogenesis | 8 | ig0.5% CMC | 598.59 ± 197.9 | 2.11 ± 0.39 | 0.67 ± 0.11 | 1.13 ± 0.28 |
| guinea pig | | | | | | |
| Normal guinea | 6 | — | 446.45 ± 98.9* | 1.35 ± 0.30*** | 0.39 ± 0.07 | 0.96 ± 0.23** |
| pig | | | | | | |

Compared with lithogenesis group
*P < 0.05
**P < 0.01
***P < 0.001

Table 2 is the result of serum glycocholic acid and all kinds of bilirubin measurement in every group of guinea pig. From the table 2, continuously taking orally the different dose of LDRS capsule, the result shows the inhibition rate of LDRS for glycocholic acid in guinea pig are respectively 31.3%, 32.0%, 35.4%. Compared with lithogenesis group, it has significant inhibition effect, and has different degree inhibition effect for all kinds of serum bilirubin, which shows LDRS has obvious antagonistic effect for the increase of glycocholic acid and bilirubin caused by gallbladder bile stasis. It has good effect for preventing bilirubin gallstone formation.

3. The influence of LDRS on the serum glycocholic acid and bilirubin Get 32 healthy female mice with body weight 28˜32 g, randomly separate them into 4 groups according to the body weight with 8 mice in each group. Forcing to take LDRS stock solution 0.154 ml/kg.d, 0.307 ml/kg.d for the 1^{st} and 2^{nd} group; Forcing to take Eulektrol 0.15 ml/kg.d for the 3^{rd} group; The 4^{th} group is blank test control group, forcing to take the same volume 0.5% carboxymethyl cellulose (abbreviated as CMC in the following). Forcing to take drug one time each day for every group, and continuously forcing to take drug for 10 days. 2 hours later after last drug-administration excise the eyeball and get blood, centrifuge 15 minutes with 3000 r/min speed, separate serum. The serum glycocholic acid and all kinds of bilirubin are measured with radioimmunoassay method and caffeine method. The result is showed in Table 3.

TABLE 3
the influence on the mouse serum glycocholic acid and
bilirubin content of LDRS and Eulektrol

| | | | glycocholic acid | serum bilirubin content (μmol/L · x ± SD) | | |
|---|---|---|---|---|---|---|
| Group | mouse | dose (ml/kg) | (μg/dl · x ± SD) | T-BIL | D-BIL | F-BIL |
| normal | 8 | ig0.5% CMC | 511.83 ± 81.3 | 1.24 ± 0.33 | 0.36 ± 0.09 | 0.83 ± 0.21 |
| blank | | | | | | |
| Eulektrol | 8 | 0.150 | 611.89 ± 135.7 | 1.45 ± 0.37 | 0.40 ± 0.14 | 1.05 ± 0.24* |
| LDRS | 8 | 0.154 | 417.86 ± 175.8^{Δ} | 0.89 ± 0.21*^{ΔΔ} | 0.23 ± 0.06**^{ΔΔ} | 0.66 ± 0.16*^{ΔΔ} |
| LDRS | 8 | 0.307 | 407.86 ± 103.3*^{ΔΔ} | 0.833 ± 0.18**^{ΔΔ} | 0.29 ± 0.06^{ΔΔ} | 0.54 ± 0.12**^{ΔΔ} |

Compared with normal blank test
*P < 0.05,
**P < 0.01
Compared with Eulektrol
^{Δ}P < 0.05
^{ΔΔ}P < 0.01,
^{ΔΔΔ}P < 0.001

From table 3, forcing to take LDRS stock solution 0.154 ml/kg.d, 0.307 ml/kg.d for 10 days has different inhibition effect for serum glycocholic acid , total bilirubin, conjugated bilirubin(direct bilirubin) and free bilirubin. Thereinto, serum glycocholic acid, total bilirubin, and free bilirubin of high dose group are significantly inhibited compared with control group, P<0.05 or 0.01, but the effect of Eulektrol is not obvious. The result is similar to the result of lithogenesis group which suggests that LDRS can decrease or reduce the increase of serum glycocholic acid, total bilirubin and free bilirubin caused by bile stasis, and has effect of lithogenesis prophylaxis and prevention.

Experiment Sample 2: Lithontriptic Experiment of LDRS

1. Gallstone Sample

1.1 Gallstone sample: All gallstones are provided by Surgery department of Sichuan people hospital, and are sandy bile duct gallstone from the same one patient. After selecting, the gallstones have uniform size, similar weight and component. The gallstone type is identified by The Chinese herb researching institute.

1.2 Bilirubin gallstone: measure the three component content of cholesterol, bilirubin and calcium.

2. Methods and Results

Get 45 autoclaved clean streptomycin vials, separate them into 3 groups with 15 vials in each group. Each vial of the 1^{st} group is loaded with about 100 mg bilirubin gallstone (with volume about 0.11±0.01 ml). Each vial of the 2^{nd} group is loaded with about 100 mg cholesterol gallstone (0.12±0.01 ml). Each vial of the 3^{rd} group is loaded with about 100 mg mixed type gallstone (0.10±0.1 ml). The gallstones of each group are all soaked with 75% ethanol for 10 minutes, then dried in the sterile glass utensil. After loading gallstone, 10 ml 0.5% carboxymethyl cellulose is added into each vial of the first 3 vials of each group. Eulektrol (0.015 ml/ml) diluted with 0.5% CMC is added into the 4^{th}˜6^{th} via¹ respectively. The cholagogue and lithontriptic solution diluted with 0.5% CMC is added into the 9 vials of 7^{th}˜9^{th}, 10^{th}˜12^{th}, 13^{th}˜15^{th}, the dose is 0.0077 ml/ml, 0.0154 ml/ml, 0.0307 ml/ml. The volume of each vial is 10 ml with PH value 7.0. The vial mouth is pricked with two-doubled gauge after drug adding. Then the vials are placed into 37° C. calorstat and kept for 60 days. Change drug solution one time every one day. Measure the residual gallstone weight [after filtering drug solution of the bottle, dry for 48 hours in 50° C., measure the total weight, then the residual gallstone weight=total weight-(gauge weight+vial weight) and its volume respectively On the 10^{th} day and 60^{th} day. The result is showed in Table 4.

TABLE 4
The lithontriptic effect of LDRS, Eulektrol to the human 3 kinds
of gallstone in vitro(x +/− SD. n = 3) f = 4

| | | | | LDRS | | |
|---|---|---|---|---|---|---|
| | Group Dose | 0.5% CMC | Eulektrol | low dose | moderate dose | high dose |
| | observation items | −10 ml | 0.015 ml/ml | 0.0077 ml/ml | 0.0154 ml/ml | 0.0307 ml/ml |
| 10 day after | bilirubin volume (ml) gallstone Residual | 0.106 ± 0.006 | 0.099 ± 0.009 | 0.913 ± 0.006 | 0.082 ± 0.001 | 0.073 ± 0.0003 |
| drug adding | weight (mg) Lithontriptic rate (%) | 96.3 ± 6.4 | 90.3 ± 9.0 | 83.0 ± 6.1 | 74.7 ± 13.3 | 66.0 ± 2.6 |
| | | 3.7 | 9.7 | 17.0 | 25.3 | 34.0 |
| | cholesterol volume (ml) gallstone | 0.12 ± 0.0 | 0.110 ± 0.012 | 0.106 ± 0.008 | 0.100 ± 0.015 | 0.086 ± 0.014 |
| | Residual weight (mg) Lithontriptic rate | 101.0 ± 6.7 | 92.7 ± 12.3 | 88.0 ± 8.3 | 84.0 ± 13 | 72.0 ± 11.0 |
| | (%) | −1.0 | 7.3 | 12.0 | 16.0 | 28.0 |
| | mixed type volume (ml) | 0.097 ± 0.004 | 0.087 ± 0.001 | 0.08 ± 0.014 | 0.08 ± 0.013 | 0.070 ± 0.006 |
| | gallstone Residual weight (mg) | 96.7 ± 3.8 | 87.3 ± 10.1 | 80.0 ± 13.8 | 76.0 ± 13.3 | 71.7 ± 6.0 |
| | Lithontriptic rate (%) | 3.3 | 12.7 | 20.0 | 24.0 | 28.3 |
| 60 day after | bilirubin volume (ml) gallstone | 0.104 ± 0.005 | 0.072 ± 0.016 | 0.064 ± 0.002 | 0.026 ± 0.001 | 0.0*** |
| drug adding | Residual weight (mg) Lithontriptic rate | 94.5 ± 5.1 | 66.0 ± 15.7 | 48.0 ± 1.7** | 22.3 ± 13.6* | 0.0 |
| | (%) | 5.5 | 34.0 | 42.0 | 77.7 | 100.0 |
| | cholesterol volume (ml) gallstone | 0.10 ± 0.014 | 0.078 ± 0.022 | 0.10 ± 0.008 | 0.09 ± 0.016 | 0.03 ± 0.002 |
| | Residual weight (mg) Lithontriptic | 96.3 ± 4.5 | 65.0 ± 22.0 | 86.5 ± 8.4 | 76.7 ± 6.2* | 24.3 ± 2.1** |
| | rate (%) | 9.7 | 35.0 | 13.5 | 23.3 | 75.7 |
| | mixed type volume (ml) gallstone | 0.09 ± 0.003 | 0.075 ± 0.005 | 0.05 ± 0.019 | 0.0356 ± 0.011 | 0.0*** |
| | Residual weight (mg) Lithontriptic | 90.7 ± 3.0 | 74.7 ± 4.6 | 50.0 ± 18.7** | 35.0 ± 10.8 | 0.0 |
| | rate (%) | 9.3 | 25.3 | 50.0 | 65.0 | 100.0 |

From table 4, three kinds of dose concentration of 0.0077 ml/ml, 0.01 54 ml/ml 0.0307 ml/ml have different degree lithontriptic effect for the three kinds of gallstone of human. The intensity of lithontriptic effect is related with the drug dose and drug administration time. When continuously administrating drug for 60 days, the lithontriptic effect of 2 dose group are significant (P<0.05 or 0.001), but the lithontriptic effect of Eulektrol is not significant.

3. Lithontriptic Experiment In Vivo

Get 32 healthy rabbits, randomly separate them into 4 groups according to the body weight and gender with 8 rabbits in each group. After grouping, iv 30 mg/kg pentobarbital sodium at the edge of ear to anaesthetise animals in each group. After anaesthesia, cut off 2×6 area long hair from the abdomen with scissors, then spray BaS depilation reagent [2] for 2 minutes, rinse thoroughly the depilation reagent with tap water, then rinse one time with normal saline to reveal the glabrous skin, then asepticize with tincture of iodine and ethanol. The glabrous skin after depilation is dissected with abdominal incision to reveal the gallbladder, to examine the gallbladder with eye to ensure without obvious abnormality, aspirate the bile, incise carefully on the top of gallbladder, implant one bilirubin gallstone and record the weight or implanted bilirubin gallstone of each rabbit. Suture the incision nick firstly, them suture the abdominal incision nick. To prevent infection, each rabbit is injected with 0.4 million unit everyday, within three days the dead rabbit is replaced. From the postoperative 3^{rd} day, drug is administrated according to the dose of each kilogram body weight. The dose are 0.2 ml/kg LDRS, 0.1 mg/mg LDRS, 0.15 ml/kg Eulektrol^{rd} group, salad oil for the 4^{th} group. Everyday infuse the drug solution into capsule, place the capsule onto the root of rabbit's tongue, then the capsule will be solely swallowed into the abdomen by the rabbit. Forcing to take medicine one time each day, continuously administrate drug for 14 days, 4 hours after last drug administration, bleeding from the femoral artery to execute the animals, dissect the gallbladder to get implanted bilirubin gallstone, absorb the drug solution and bile with filter paper, make the best to find the gallstone, measure the residual gallstone weight after the weight is constant at 60° C. constant temperature, compared with the control group.

TABLE 5
The influence of continuously orally take different dose LDRS
to the implanted human bilirubin gallstone of the rabbit

| | | | The gallstone weight | The residual weight | |
|---|---|---|---|---|---|
| | Animals | dose | before litholysis | after litholysis | Decreasing |
| Group | number | ml · (kg · d)⁻¹ | (mg · x ± SD) | (mg · x ± SD) | rate (%) |
| LDRS | 8 | 0.1 | 96.0 ± 3.3 | 45.45 ± 11.15 | 52.6 |
| LDRS | 8 | 0.2 | 96.0 ± 1.72 | 38.01 ± 11.76 | 60.4 |
| Eulektrol | 8 | 0.15 | 95.2 ± 2.32 | 79.83 ± 10.91 | 22.82 |
| salad oil | 8 | 0.15 | 94.4 ± 3.35 | 86.80 ± 24.24 | 7.9 |

Compared with salad oil group
***P < 0.001;

From Table 5, human bilirubin gallstone is implanted into the rabbit, continuously forcing to take different dose LDRS for 14 days has different degree litholysis, the weight of implanted gallstone is decreased. The decreasing rate is 52.6%, 60.4% respectively. Compared with the control group, the decreasing rate is very significant, P<0.001: but Eulektrol group also has different degree decreasing, the decreasing rate is 22.8%, compared with the control group, the decreasing is not significant, P<0.05. It shows LDRS has certain lithontriptic effect to the bilirubin gallstone.

Experiment Sample 3: Cholagogue Experiment of LDRS in Rat

Get 70 healthy male rat with body weight 280˜410 g, separated them into 7 groups, the grouping condition is showed in Table 5. After grouping, the experimental rats are fasting for 16 hours before experiment; anesthetize these rats through ip ethyl carbamate (also named Urethane). After anesthesia, enlace the four limbs; incise the abdomen to find the pylorus of the stomach. Along the pylorus, find common bile duct. Use little forceps to deliminate the surface envelope and reveal the common bile duct, ligate the lower extremity, carefully shear a little ventage, insert plastic duct with external diameter 1 mm into the common bile duct proximal to the liver to drain the bile. After bile drainage volume is stable, record the bile collection volume every 0.5 hour, for two times. Administrate the drug or control solution 2 ml/100 g.bw into duodenum, measure the bile efflux coefficient of every zone 4 hours later after drug administration, compare the bile secretion coefficient with control group (bile secretion coefficient=actual bile collection volume/b.w.×100%), the result is showed in Table 6.

TABLE 6
the compare of bile efflux coefficient between different dose
LDRS and positive-effect cholagogue drug

| | | the variation of bile efflux coefficient before drug administration | | | | | | | | |
|---|---|---|---|---|---|---|---|---|---|---|
| | | and after drug administration ml(bile)/100 g · bw | | | | | | | | |
| | dose | before | | | | | | | | |
| Group | (ml/kg) | 1 hour | after 1 hour | (%) | after 2 hours | (%) | after 3 hours | (%) | after 4 hours | (%) |
| Blank control | Ig same | 0.116 ± 0.04 | 0.156 ± 0.04 | — | 0.149 ± 0.07 | | 0.110 ± 0.09 | | 0.113 ± 0.06 | |
| | control | | | | | | | | | |
| | solution | | | | | | | | | |
| LDRS | 0.0163 | 0.116 ± 0.04 | 0.197 ± 0.08 | 26.2 | 0.205 ± 0.04* | 37.6 | 0.205 ± 0.04** | 86.4 | 0.171 ± 0.08 | 51.3 |
| LDRS | 0.0325 | 0.116 ± 0.04 | 0.233 ± 0.11* | 49.4 | 0.270 ± 0.01*** | 81.2 | 0.210 ± 0.08* | 91.0 | 0.192 ± 0.09* | 69.9 |
| LDRS | 0.0650 | 0.116 ± 0.04 | 0.235 ± 0.09* | 51.0 | 0.300 ± 0.04*** | 101.3 | 0.250 ± 0.09*** | 127.0 | 0.199 ± 0.08** | 76.1 |
| Eulektrol | 0.150 | 0.116 ± 0.04 | 0.145 ± 0.04 | −7.1 | 0.144 ± 0.04 | −3.4 | 0.138 ± 0.04 | 25.5 | 0.128 ± 0.04 | 13.3 |
| Cholanic acid | 100 mg (pure | 0.116 ± 0.04 | 0.166 ± 0.04 | 6.4 | 0.189 ± 0.05 | 26.8 | 0.246 ± 0.04*** | 123.6 | 0.164 ± 0.04* | 45.1 |
| | substance) | | | | | | | | | |
| Dehydrogenized | 100 mg (pure | 0.116 ± 0.04 | 0.165 ± 0.08 | 5.8 | 0.234 ± 0.07* | 57.0 | 0.223 ± 0.05** | 102.7 | 0.210 ± 0.07** | 85.8 |
| Cholanic acid | substance) | | | | | | | | | |

Compared with the control group,
*P < 0.05,
**P < 0.01,
***P < 0.001

From Table 6, 0.0163 ml, 0.0325 ml, 0.0650 ml/kg LDRS stock solution, 0.15 ml/kg Eulektrol, 100 mg/ml cholanic acid, 100 ml/kg dehydrogenized cholanic acid are all administrated from the duodenum of the experimental rats. 1 hour later after drug administration, compared with control group, the bile secretion coefficient of each group is respectively increased 26.2%, 49.4%, 81.2%, 101.3%, −3.4%, 26.8%, 57.0%; 3 hours later, respectively increased 86.4%, 91.0%, 127.3%, 102.7%. And 4 hours later, compared to the control group still respectively increased 51.3%, 69.9%, 76.1%, 13.3%, 45.1%, 85.8%. From the aforementioned result, highs moderate and low dose LDRS capsule all have obvious cholagogue effect, and the effect is obviously dependent on the dose, the cholagogue effect can last for more than 4 hours. Although Eulektrol has certain cholagogue effect, but it acts slowly and the bile secretion coefficient is increased only after 3 hour after drug administration, which shows Eulektrol acts slowly; Although the cholagogue effect of cholanic acid and dehydrogenized cholanic acid is verified, but the action time of cholagogue effect is slow than LDRS, it acts 2 hours after drug administration, thereinto the cholagogue effect of dehydrogenized cholanic acid can last longer time, but the cholagogue effect of cholanic acid acts slowly and lasts shorter time. The experimental results shows: LDRS has stronger cholagogue effect.

Experiment Sample 4: The Measure of Bilirubin of Bile

TABLE 7
The influence of different dose cholanic acid, Eulektrol, dehydrogenatedcholanic acid,
LDRS to all kinds of bilirubin in bile

| group | control group | cholanic acid | Eulektrol | Dehydrogenized | High dose | Moderate dose | Low dose |
|---|---|---|---|---|---|---|---|
| dose(ml/kg) | ig same CMC | 0.1(g) | 0.15(g) | cholanic acid 0.1(g) | LDRS 0.615 | LDRS 0.307 | LDRS 0.154 |
| O T-BIL | 92.3 ± 37.1 | 92.6 ± 33.4 | 91.8 ± 32.7 | 92.2 ± 31.9 | 93.0 ± 33.4 | 92.7 ± 32.7 | 92.0 ± 30.9 |
| (h) D-bil | 50.7 ± 27.7 | 51.8 ± 25.6 | 52.0 ± 24.7 | 50.9 ± 23.3 | 50.9 ± 28.7 | 51.5 ± 24.8 | 51.0 ± 27.8 |
| F-bil | 41.6 ± 24.8 | 40.8 ± 23.6 | 39.8 ± 22.8 | 41.3 ± 22.9 | 42.2 ± 23.8 | 41.2 ± 22.6 | 41.0 ± 21.9 |
| 1 T-BIL | 93.2 ± 57.1 | 94.1 ± 37.9 | 104.1 ± 50.0 | 73.1 ± 14.1 | 55.0 ± 23.2 | 69.7 ± 20.2 | 82.4 ± 27.2 |
| (h) D-bil | 51.8 ± 32.3 | 67.3 ± 25.4 | 81.7 ± 35.9 | 57.8 ± 12.8 | 38.1 ± 15.7 | 51.1 ± 20.4 | 55.6 ± 20.5 |
| F-bil | 41.4 ± 22.3 | 26.7 ± 20.1 | 20.9 ± 17.7* | 15.4 ± 4.9** | 19.4 ± 14.2* | 18.6 ± 14.9* | 26.7 ± 12.5* |
| 2 T-BIL | 98.8 ± 37.2 | 77.7 ± 38.9 | 81.90 ± 27.0 | 54.3 ± 23.9* | 41.0 ± 20.0** | 68.8 ± 16.1* | 77.8 ± 25.6 |
| (h) D-bil | 56.7 ± 19.2 | 60.8 ± 31.9 | 69.3 ± 27.4 | 43.4 ± 15.5 | 25.1 ± 13.1** | 47.1 ± 10.9 | 54.1 ± 15.6 |
| F-bil | 42.6 ± 22.6 | 16.9 ± 12.8* | 12.6 ± 5.1 | 10.9 ± 11.2* | 15.9 ± 11.3** | 23.5 ± 11.9* | 23.7 ± 16.8 |
| 3 T-BIL | 138.9 ± 91.6 | 82.5 ± 29.0 | 74.3 ± 32.7 | 48.1 ± 14.8* | 46.7 ± 20.6* | 69.0 ± 29.5* | 74.6 ± 47.2 |
| (h) D-bil | 62.5 ± 34.3 | 55.3 ± 18.2 | 59.0 ± 18.2 | 39.9 ± 10.2 | 23.9 ± 9.6** | 48.4 ± 19.0 | 41.8 ± 21.8 |
| F-bil | 76.3 ± 61.0 | 27.2 ± 13.9* | 17.3 ± 12.9 | 8.16 ± 3.1** | 19.0 ± 11.9* | 20.6 ± 17.4* | 32.8 ± 26.3 |
| 4 T-BIL | 171.6 ± 124 | 81.3 ± 29.8* | 78.5 ± 31.8 | 59.3 ± 19.7* | 45.2 ± 19.7* | 82.5 ± 46.0 | 91.7 ± 56.8 |
| (h) D-bil | 79.0 ± 42.4 | 53.0 ± 31.6 | 57.7 ± 14.9 | 45.7 ± 16.4 | 26.7 ± 7.6** | 53.9 ± 25.7 | 49.7 ± 24.3 |
| F-bil | 92.6 ± 87.4 | 28.3 ± 10.6* | 20.8 ± 22.3 | 13.6 ± 5.69* | 18.8 ± 8.34* | 28.6 ± 21.1* | 42.0 ± 23.3 |

Compared with control group
*p < 0.0,
**P < 0.01 (n = 8, BIL unit: mmol/L X ± SD)

From table 7, different dose cholanic acid, dehydrogenated cholanic acid and LDRS groups have different degree inhibition effect to the total bilirubin, conjugated bilirubin and free bilirubin in bile. Thereinto, high dose LDRS and dehydrogenated cholanic acid have significant inhibition effect to the conjugated bilirubin and free bilirubin, and have significant difference with control group, P<0.05, P<0.01.

Experiment sample 5: The therapeutic effect of LDRS to the mouse infected by staphylococcus aureus.

Experimental method: preparation of bacteria solution, get clinically separated staphylococcus aureus, inoculated in the tube with beef soup liquid culture medium, cultured for 16˜18 hours in 37° C. incubator. Then dilute the cultured bacteria solution with normal saline with 10-2 ratio, add double volume 5 g/dl gastrin suspension to intensify the bacterial virulence for infecting the mouse.

Get 40 mice with 18˜22 g body weight separate them into 4 groups according to body weight and gender with 10 mice in each group (5 female mice and 5 male mice), to design three groups with highs moderates low dose, one control group without drug administration. According to the bacteria suspension concentration to make 90% mice dead which is determined through the pilot experiment, inject about 0.2 ml/10 g.bw bacteria suspension concentration to each experimental mouse through the abdomen cavity injection pathway, which is relative to 5×10⁹ bacteria/each mouse. 1˜2 hours before infection, mice in each experimental group are all forced to take LDRS 0.077 ml, 0.154 ml, 0.307 ml/kg, mice in control group are all forced to take 0.5% CMC. After infection, administrate drug once again. Afterwards force to take drug one time every day, continuously force to take drug for 5 days. Examine the growth and death condition of mice every day. Use the survival rate in the 7th day after infection as examination indicator. The result is showed in Table 8.

TABLE 8
The therapeutic effect of LDRS to the mouse infected by
staphylococcus aureus

| | | | | survival rate |
|---|---|---|---|---|
| | | | | (survival |
| | | | | mice |
| | | | | number/ |
| | | | | experimental |
| | experimental | drug dose | dead mice | mice |
| Group | mice(number) | (ml/kg) | (number) | number) |
| LDRS | 10 | 0.077 | 6 | 4/10 |
| LDRS | 10 | 0.154 | 3** | 7/10* |
| LDRS | 10 | 0.307 | 1*** | 9/10*** |
| Infection group | 10 | Ig same | 9 | 1/10 |
| without drug | | voulme0.5% | | |
| administration | | CMC | | |

Compared with infection group,
*P < 0.05,
***P < 0.00

From Table 8, 2 hours before infection on each day force to take different dose LDRS and continue 5 days, examine for 7 days, the result shows that LDRS has different degree increase to the survival rate of mice, and has obvious dose-effect relation. Low, moderates high dose LDRS are respectively higher 30%, 60%, 80% compared with infection group without drug administration, and all have therapeutic effect to the staphylococcus aureus. Thereinto, the moderates high dose group are compared with control infection group without drug administration, P<0.05 and P<0.001 respectively. The low dose group has not significant difference compared with control group, P>0.05 because of a smaller sample, but it increase 30% to the survival rate. The result tallies with the experimental result of experiment in vitro. Experiment sample 9: The influence of LRDS to the gallbladder smooth muscle contraction caused by acetylcholine.

Get 9 guinea pigs fasting 24 hours with body weight more than 300 g, exsanguinate the guinea pig after knockout, anatomize the abdomen to reveal gallbladder, suture the base and neck of gallbladder with one thin suture respectively, sever the common bile duct after ligating, carefully take out the gallbladder, place it into 4° C. Krebs liquid culture plate, anatomize longitudinally to get 2 pieces of muscle stripe of gallbladder with 10 mm length and Btam width. Place the muscle stripe into 37° C. constant temperature Mcrruary bath with Kmbs solution (20 ml), the lower end is fixed on the hook at the bottom of the bath, the upper end is connected to the energy transducer, adjust the oxygen flow gas bubble to tiny and uniform flow, the energy transducer is connected to physiological recorder with prepositive amplifier. Every time after adding test solution observes for more than 3 minutes. When reaching the biggest effect, timely rinse the bath with flushing solution three times with 15 minutes break. After the contraction curve becomes balanced, to make another experiment. In each experiment, we can add 0.2-0.4 ml 0.1 ug/ml acetylcholine to observe if there is gallbladder muscle stripe contraction action response.(what we should pay attention to in the experiment are below: prepare the gallbladder muscle stripe rapidly and carefully without lacerating; Krebs solution should be prepared with double-distilled water). After adding acetylcholine, record the contraction curve of gallbladder muscle. From the contraction curve, we can see the amplitude of contraction curve rise. After contraction curve reaches peak-balance, add different concentration LDRS solution. After adding drug solution, the amplitude of contraction curve of gallbladder muscle stripe descends. Record the altitude of amplitude of every phase. The statistic method is below: before drug administration, from the upper and lower limit point of the amplitude contraction curve of gallbladder muscle stripe to respectively line a straight line parallel to the transverse coordinate axis, measure the altitude between the two parallel line as the normal amplitude (The lower line before drug administration is used as basal line). After contraction curve is stable, adding different concentration Ach solution, we can see the amplitude of contraction curve of gallbladder muscle stripe rise, through the peak of amplitude to line a straight line parallel to basal line and measure the altitude between the two parallel line as amplitude of Ach. After curve is stable, add different concentration LDRS stock solution and through the lowest point of amplitude to line parallel to basal line, measure the altitude between the basal line and the lowest line. The result is showed in Table 9.

TABLE 9
the influence of LDRS to the gallbladder contraction
amplitude caused by Ach

| | Dose | number of | amplitude | variation |
|---|---|---|---|---|
| Group | (Concentration) | muscle stripe | (mm · x ± SD) | rate (%) |
| Normal | basal culture | 13 | 6.5 ± 2.2*** | 100.0 |
| value | medium | | | |
| Ach | 10⁻⁴ ml | 13 | 35.2 ± 6.84 | 541.5 |
| LDRS | 10⁻² ml | 13 | 13.4 ± 6.90*** | 206.2 |
| Normal | basal culture | 8 | 5.0 ± 3.2*** | 100.0 |
| value | medium | | | |
| Ach | 10⁻⁴ ml | 8 | 32.73 ± 6.90 | 654.6 |
| LDRS | 10⁻² ml | 8 | 16.30 ± 7.30** | 326.0 |

Compared with Ach
**P < 0.01,
***P < 0.001

From Table 9, Adding different concentration LDRS 10⁻², 10⁻³ can make the altitude of the amplitude of Ach⁻⁴ respectively decrease 61.9%, 50.2%, which shows that LDRS has significant inhibition effect to the increase of amplitude of gallbladder muscle stripe in vitro caused by Ach, compared with the altitude of amplitude of Ach group P<0.01, P<0.001, showing good spasmolytic effect.

Experiment sample 10: The influence of LDRS to the intestinal muscle in vitro of guinea pig.

Get 3 male guinea pigs fasting for 24 hour, anatomize the abdomen after knockout to get many ileum segment with 3-4 cm length, wipe off the adipose and membrane tissue attached to the external surface of ileum, place them into 5% CO₂, 37° C. preserving fluid, clean up the content, to prepare into intestine segment with 1.5 cm length, respectively suture a filament on the two ends of intestine segment, one end is hanged on the L-shape sustaining pole and placed into bath, the other end is connected with energy transducer and applied 1.0 g stress. After recording the contraction curve of intestine through two-channel physiological recorder(LMS-2B model made by ChengDu instrument factory), in order add the following drug into the nutrition solution: 1:10000 Ach 0.05 ml, 1:1000 histamine, 1:1000 BaCl₂, the intestinal muscle is spasmic at once, the amplitude of intestinal muscle obviously increase. When recording the amplitude peak of contraction curve, add the experimental drug LDRS, the result is showed in FIG. 1. From the FIG. 1, LDRS has obvious inhibition effect to the contraction of normal intestine muscle, and can relax the spasm caused by Ach, BaCl₂, HT, showing the intestine muscle relax function.

Experiment Sample 11: The Anti-Inflammation Experiment of LDRS

1. The influence result to the auricular inflammation of mouse caused by dimethylbenzene is showed in Table 10.

TABLE 10
The influence of LDRS, indomethacin, Eulektrol
to the auricular weight of mouse

| | | | the auricular | |
|---|---|---|---|---|
| | | | weight difference | |
| | | | between left | |
| | drug dose | mice | and right (mg/ | inhibition rate |
| Group | (ml/kg) | number | 100 g · x ± SD) | (%) |
| 0.5% CMC | 10 | 10 | 15.33 ± 3.3 | — |
| Eulektrol | 0.15 | 10 | 12.30 ± 4.3 | 14.5 |
| Indothemacin | 30 (mg) | 10 | 3.98 ± 2.5 | 74.0 |
| LDRS | 0.077 | 10 | 8.50 ± 4.1** | 44.5 |
| LDRS | 0.154 | 10 | 5.60 ± 3.8*** | 63.4 |
| LDRS | 0.307 | 10 | 4.90 ± 2.3*** | 68.0 |

Compared with 0.5% CMC
**P < 0.01
***P < 0.001

From Table 10, LDRS and Eulektrol can all inhibit the auricular swollen of mouse caused by dimethylbenzene, but the inhibition degree is less than indomethacin, and compared with control group have significant inhibition effect, P<0.0 1, P<0.00 1, and have obvious dose-effect relation.

2. The influence result of LDRS to the granuloma of rat caused by agar is showed in Table 11.

TABLE 11
The influence of LDRS, Prednisone acetate to the
granuloma weight caused by cotton

| | experimental | | average granuloma | inhibition |
|---|---|---|---|---|
| | rats | drug dose | coefficient | rate |
| Group | number | (ml/kg) | (^{α} · x ± SD) | (%) |
| 0.5% CMC | 10 | 10 | 0.65 ± 0.47 | — |
| Prednisone | 10 | 30 (mg) | 0.20 ± 0.08** | 69.2** |
| LDRS | 10 | 0.077 | 0.44 ± 0.19 | 32.3 |
| LDRS | 10 | 0.154 | 0.32 ± 0.17* | 50.7* |
| LDRS | 10 | 0.307 | 0.23 ± 0.05** | 64.6* |

Compared with 0.5% CMC
*P < 0.05
**P < 0.01

From Table 11, continuously forcing to take different dose LDRS stock solution 0.077 ml, 0.154 ml, 0.307 ml/kg.d for 10 days has inhibition rate of 32.3%, 50.7%, 64.6% respectively for the granuloma coefficient of rat caused by cotton, which shows forcing to take LDRS has different degree inhibition effect to the granuloma of rat caused by agar. Thereinto, the inhibition effect of moderates high dose group is compared with control group, P<0.05, P<0.01 respectively, showing significant inhibition effect. The inhibition effect of high dose group is compared with prednisone group, P>0.05, showing no significant difference, suggesting that the content of soft capsule has good anti-inflammation effect to the chronic inflammation.

3. The experimental anti-inflammation effect result of LDRS to the ankle swollen of the rat is showed in Table 12

TABLE 12
The influence of different dose LDRS, Eulektrol, prednisone
acetate to the ankle swollen caused by carragheen

| | Experimental | Drug dose | size of ankle(cm · X +/− SD) | | | | | |
|---|---|---|---|---|---|---|---|---|
| Group | rats number | (ml/kg) | 0 h before drug | 2 h after drug | 4 h after drug | 8 h after drug | 16 h after drug | 20 h after drug |
| 0.5% CMC | 9 | 1.10 ± 0.08 | 1.93 ± 0.89 | 2.78 ± 0.25 | 3.54 ± 0.23 | 2.55 ± 0.19 | 2.31 ± 0.18 | 2.11 ± 0.19 |
| Eulektrol | 8 | 0.15 | 2.01 ± 0.19 | 2.70 ± 0.19 | 2.90 ± 0.16*** | 2.70 ± 0.15 | 2.40 ± 0.12 | 2.00 ± 0.12 |
| Prednisone | 8 | 30(mg) | 1.98 ± 0.19 | 2.34 ± 0.17*** | 2.10 ± 0.15*** | 1.90 ± 0.19*** | 2.10 ± 0.211 | 2.00 ± 0.009 |
| acetate | | | | | | | | |
| LDRS(low) | 9 | 0.077 | 1.96 ± 0.19 | 2.70 ± 0.19 | 2.60 ± 0.21**** | 2.30 ± 0.11 | 2.20 ± 0.11 | 2.09 ± 0.11 |
| LDRS(moderate) | 8 | 0.154 | 1.97 ± 0.15 | 2.65 ± 0.16 | 2.05 ± 0.45*** | 2.11 ± 0.12*** | 2.10 ± 0.10* | 2.06 ± 0.09 |
| LDRS(high) | 9 | 0.307 | 1.99 ± 0.16 | 2.55 ± 0.17* | 2.15 ± 0.09*** | 2.16 ± 0.16*** | 2.10 ± 0.16* | 2.00 ± 0.12 |

Compared with 0.5% CMC
*P < 0.05
**P < 0.01
***P < 0.001

From table 12, LDRS stock solution 0.077 ml, 0.154 ml, 0.307 ml/kg.d and prednisone acetate 30 mg/kg all have good inhibition effect for the ankle swollen of rat caused by carragheen, compared with 0.5% CMC, at 2hours and 4 hours after drug administration have significant inhibition effect. High dose LDRS is compared with prednisone acetate P>0.05, without significant difference, which shows the content of soft capsule has good anti-inflammation effect to PG type inflammation.

Experiment 12: The influence of LDRS to the foot-licking response of mouse caused by 2.5% formaldehyde.

Get 60 healthy male mice with body weight 18-30 g, randomly separate them into 6 groups according to the body weight. The mice in the 1^{st} group are forced to take 0.5% CMC 10 ml/kg. SC morphine hydrochloric acid 10 mg/kg for the 2^{nd} group; Eulektrol 0.15 ml/kg for the 3^{rd} group, LDRS 0.077 ml, 0.154 ml, 0.307 ml/kg.d. In every group, after the first time of forcing to take drug, SC 2.5% formaldehyde 0.03 ml/mose on the dorsal surface of the right rear foot. Then after injection, record the foot-licking counts at once for 15 minutes, the result is showed in Table 13.

TABLE 13
The influence of Eulektrol, morphine to the foot-licking
counts of mice caused by 2.5% formaldehyde

| | | | Mice foot-licking | Inhi- |
|---|---|---|---|---|
| | | | counts after SC | bition |
| | Experimental | Drug dose | 2.5% formaldehyde | rate |
| Group | mice number | (ml/kg) | (Number · X +/− SD) | (%) |
| 0.5% CMC | 10 | 10 | 75.375 ± 9.257 | — |
| Morphine | 10 | 10 (mg) | 1.2860 ± 2.630*** | 98.3 |
| Eulektrol | 10 | 0.15 | 72.780 ± 9.670ΔΔΔ | 3.4 |
| LDRS (low) | 10 | 0.077 | 40.750 ± 9.489***ΔΔΔ | 45.7 |
| LDRS | 10 | 0.154 | 25.00 ± 9.303***ΔΔΔ | 66.8 |
| (moderate) | | | | |
| LDRS (high) | 10 | 0.307 | 10.125 ± 6.128***ΔΔΔ | 86.6 |

Compared with 0.5% CMC
***P < 0.001;
compared with morphine group
ΔΔΔP < 0.001

From table 13, LDRS can significantly decrease the foot-licking times of mouse, demonstrate that LDRS has good analgesic effect. In the positive control group, the inhibition rate of morphine to the foot-licking counts caused by formaldehyde is more than 98%, but the analgesic effect of Eulektrol is not obvious (P>0.05). The analgesic intensity of LDRS is increasing with the increase of the dose, and shows good dose-effect relation, but the analgesic intensity is weaker than morphine. The inhibition rate of high dose LDRS is compared with morphine P<0.001, with significant difference.

Experiment sample 13: the influence of LDRS to the fever of the rat caused by 2, 4-dinitrophenol.

Get 49 healthy male rats with 1 80˜220 g body weight, randomly separate them into 6 groups according to the body weight with 8 rats in each group. The rats in the 1^{st} group are forced to take 0.5% CMC 10 ml/kg. The rats in the 2^{nd} group are forced to take aminopyrine 80 ml/kg. The rats in 3^{rd} group are forced to take Eulektrol 0.15 ml /kg. The rats in the 4^{th}, 5^{th}, 6^{th} are respectively forced to take LDRS 0.077 ml, 0.154 ml, 0.307 ml/mg. 15 minutes later after drug administration SC 25 mg/ml 2,4-dinitrophenol in the middle of shoulder and back, then measure the anal temperature in 0 hour before drug administration, 0.25 hours, 0.5 hours, 1 hours, 2 hours, 4 hours after drug administration. The result is showed in Table 14.

TABLE 14
The influence of LDRS, aminopyrine, Eulektrol on the fever of rats caused by 2,4-dinitrobenzene

| | Experimental | drug dose | before drug | the body temperature variation after drug administration(° C. Δ X ±/− SD) | | | | |
|---|---|---|---|---|---|---|---|---|
| Group | rats number | (ml/kg) | administration 0 h | 0.25 h | 0.5 h | 1 h | 2 h | 4 h |
| 0.5% CMC | 8 | 10 | 36.05 ± 0.29 | 1.05 ± 0.76^{ΔΔ} | 2.66 ± 0.59^{ΔΔ} | 2.91 ± 0.77^{ΔΔ} | 1.78 ± 0.67^{ΔΔ} | 0.69 ± 0.78^{Δ} |
| Aminopyrine | 8 | 80(mg) | 36.31 ± 0.43 | −0.21 ± 0.52** | −0.05 ± 0.81** | 0.03 ± 0.79** | 0.49 ± 0.47** | 0.30 ± 0.67 |
| Eulektrol | 8 | 0.15 | 36.25 ± 0.31 | 0.24 ± 1.01 | 1.51 ± 0.70** | 1.08 ± 0.55** | 1.28 ± 0.53 | 0.42 ± 0.60 |
| LDRS(low) | 8 | 0.077 | 36.52 ± 0.15 | −0.08 ± 0.47** | 0.68 ± 0.55** | 0.13 ± 0.33** | 0.51 ± 0.56** | 0.33 ± 0.48* |
| LDRS(moder- | 8 | 0.154 | 36.3 ± 0.41 | −0.06 ± 0.66** | 0.39 ± 0.73** | 0.61 ± 0.79** | 0.48 ± 0.47** | 0.17 ± 0.88 |
| ate) | | | | | | | | |
| LDRS(high) | 9 | 0.307 | 36.4 ± 0.31 | −0.2 ± 0.64** | −0.29 ± 0.62** | 0.14 ± 0.71*** | 0.14 ± 0.50** | −0.39 ± 0.56** |

Note:
0.5% CMC˜14: compared with the effect before drug administration,
^{Δ}denotes P < 0.05,
^{ΔΔ}denotes P < 0.01
In the same time after drug administration, compared with 0.5% CMC,
*denotes P < 0.05,
**denotes P < 0.01.

From Table 14, the effect of forcing to take different dose LDRS is similar to that of aminopyrine and have obvious inhibition effect to the fever of rat caused by 2, 4-dinitrophenol, the intensity of inhibition is relative to dose.

The invention can make the aforementioned effect come true for the experimental samples.

Experimental Sample 1:

Mint Oil 320 ml Herit Oil 80 ml

According to the recipe proportion, mix uniformly the aforementioned 2 kinds of Chinese herb and prepare them into 1000 soft capsules with 0.4 ml in each capsule.

Experimental Sample 2:

Mint Oil 300 ml Herit Oil 100 ml

According to the recipe proportion, mix uniformly the aforementioned 2 kinds of Chinese herb and prepare them into 1000 dropping pills with 0.4 ml in each pill.

Experimental Sample 3:

The quality control method for soft-capsule-form drug dose of the invention:

Identification: Get 1 drop of soft capsule content, add 3-5 drops of H₂SO₄ and a little amount of vanillin crystal, it should be orangered, then add 1 drop of water, it should be violet;

Get 1 ml of soft capsule content, add 10 ml acetoacetate, then mix them as test solution; Additionally get mint with acetoacetate to prepare the control mint solution with 1 mg/ml concentration. According to the thin-layer chromatography experiment, (Appendix. VIB, Volume I, Chinese Pharmacopoeia, edition in 2000), aspirate 2 ul from each of the aforementioned three kind of solution respectively, drop them onto the same silica-gel G thin-layer plate, use 15:3 petroleum ether-acetoacetate at 30-60° C. as developer, thin-layer plate, use 15:3 petroleum ether-acetoacetate at 30-60° C. as developer, develop them below 25° C., then get them out and drying them, spray with newly prepared 5% vanillin sulphate solution, blow with hot wind until the dot is clear. The chromatogram of the test solution has the same color dot in the corresponding position with the control solution chromatogram;

Relative density: get soft capsule content, measure the relative density (picnometer method, Appendix VII A, Volume I, Chinese Pharmacopoeia, edition in 2000) according to the relative density assay method, the relative density should be 0.881˜0.901;

Refractive index: get soft capsule content, measure refractive index legally (Appendix VII F, Volume I, Chinese Pharmacopoeia, edition in 2000), the refractive index should be 1.456˜1.466;

Specific rotation: get content as the relative density measurement, measure specific rotation legally (Appendix VII E, Volume I, Chinese Pharmacopoeia, edition in 2000), the specific rotation should be −14°˜−22°;

Content Measurement:

The Measurement of Ester Content and Total Alcohol Content

Get soft capsule content about 5 g, quantify minutely, place them into the flask, add 10 ml ethanol which is indicated neutral by phenolphthalein indicator solution, mix them, and add 2 drops of phenolphthalein indicator solution. At first, neutralize the free acid with KOH solved in 0.1 mol/L ethanol, then add minutely 25 ml 0.5 mol/L KOH volumetric solution solved in ethanol, reflux for 0.5-1.5 hours in water bath, cool them, then add 0.5 ml phenolphthalein indicator solution, use 0.5 mol/L HCL to slowly titrate the residual KOH, make the blank test at the same time. 1 ml 0.5 mol/L KOH volumetric solution correspond to 99.15 mg menthol acetate(C₁₂H₂₂O₂); through the corresponding mass of the menthol acetate, the ester content of each soft capsule content should be 5-11% (g/g);

Acetylation: get 10 ml soft capsule content, place them into acetylating flask with 100 ml volume, add 10 ml acetic acid and 2 g newly solved anhydrous sodium acetate, with air condenser attached to the flask, placed into 145±3° C. oil bath or sand bath, boiled for 0.5-1.5 hours, get them out and cool, through condenser add 50 ml, place into water bath, vibrate, heat for 10-20 minutes, then cool them; placed into dispenser funnel, get rid of the underlayer acidic solution; add 50 ml saturated NaCl solution, vibrate fully, stationary fractionation, get rid of upper layer water solution, then wash with water for several times, 50 ml every time, until the washing solution is indicated neutral by phenolphthalein indicator solution; place the acquired acetylating oil into 25 ml Erlenmeyer flask with plug, add 3 g anhydrous sodium sulphate, close the flask tightly with the plug, and vibrate ever and agah, make dehydration until when getting 1 drop of acetylating oil mixed with 10 drops of CS₂ no turbidity occurs, then filtrate with dry filter paper;

Saponification: get about 1 g dry acetylating oil, quantify minutely, place into 100 ml Erlenmeyer flask, add 25 ml KOH solution solved in 0.5 mol/L ethanol, reflux for 0.5-1.5 hours in water bath, cool, take the condenser away, drop 0.5 ml phenolphthalein indicator, use 0.5 mol/L HCL solution titrate the residual KOH, make the blank test at the same time. Calculate as the following formula, we can get the result:

$$Total alcohol content \% = \frac{( B - A ) \times N \times 0.1543}{W - ( B - A ) \times N \times 0.04204} \times 100 \%$$

B: the volume of 0.5 mol/L HCl standard solution consumed in the blank test (ml);

A: the volume of 0.5 mol/L HCL standard solution consumed by acetylating oil (ml);

N: molar concentration of standard solution;

W: mass of the acetylating oil (g);

0.1543: the milligram molar number of geraniol;

0.04202: the milligram molar number difference between acetate and ketol;

Through the corresponding mass of geraniol, the total alcohol content of each soft capsule should not be less than 50.0%.

The Measurement of Menthol and Geraniol Content

Referring to the gas chromatography test method (page 40, Appendix VI E, Volume I, Chinese Pharmacopoeia, edition in 2000)

chromatography condition and system adaptability experiment, capillary chromatographic column: 5% diphenyl-95% dimethyl silicane copolymer as 30 m×0.32 mm×0.25 μm solid capillary chromatographic column; sample inlet temperature: 240° C.; detector temperature: 240° C.; make sample measurement with the distribution ratio of 50:1; heat-up progressively, initial temperature 80° C., maintain for 2 minutes, heat-up to 170° C. with the heat-up speed 8° C. every minute, maintain for 3 minutes; flow rate of carrier gas: 1.5 ml/min; According to the geraniol peak theoretical plate number should not be less than 300000;

Correction factor measurement get moderate n-pentadecane or n-tridecane or n-tetradecane or naphtalene or camphor or n-undecane etal as internal standard control sample, measure precisely, add acetoacetate to prepare into 2.5 mg/ml concentration solution as internal standard solution; Get adequate amount of menthol geraniol control sample, measure precisely, add acetoacetate to prepare into solution menthol 6 mg/ml concentrations geraniol 1 mg/ml concentration, aspirate precisely aforementioned three kinds of solution 2 ml respectively, placed into 10 ml quantifying flask, add acetoacetate, dilute it to the scale, vibrate uniformly, aspirate 1 ul, injected into gas chromatograph, measure, measure the correction factor;

Get about 50 mg soft capsule content of loading difference sample, measure precisely, placed into 10 ml quantifying flask, add 2 ml internal standard solution minutely, add acetoacetate, dilute it to the scale, vibrate uniformly, aspirate 1 ul, injected into gas chromatograph, measure, then we can acquire: according to the menthol(C₁₀H₂₀O), the mint oil in each soft capsule content should not be less than 79 mg; according to the geraniol(C₁₀H₁₈O), the herit oil should not be less than 7 mg.

1. A kind of drug complex, with the feature that the drug complex contains any volume ratio mint oil and herit oil.

2. A kind of drug complex as stated by claim 1, with the feature that the drug complex contains drug with the following volume ratio: mint oil:herit oil 2-6:1-2.

3. A kind of drug complex as stated by claim 1, with the feature that the drug complex contains drug with the following volume ratio: mint oil:herit oil 3:1.

4. A kind of drug complex as stated by claim 1, with the feature that the drug complex contains drug with the following volume ratio: mint oil:herit oil 4:1.

5. A kind of drug complex as stated by claim 1, 2, 3 or 4, with the feature that the drug complex can be added into the conventional accessories and be prepared into the clinically accepted dose type, such as pill, tablet, orally-take liquid preparations capsules granule form of prepared drug.

6. The application of the drug complex as stated by claim 1, 2, 3 or 4 in the forms of prepared drug with lithogenesis prophylaxis and gallstone therapy effect.

7. The application as stated by claim 6, with the feature that the lithogenesis prophylaxis and gallstone therapy effect refers to inhibiting the increase of glycocholic acids, total bilirubin and free bilirubin.

8. The application of the drug complex as stated by claim 1, 2, 3 or 4 in the form of prepared drug with lithontriptic effect.

9. The application of the drug complex as stated by claim 8, with the feature that lithontriptic effect for gallstone refers to dissolving the bilirubin gallstone.

10. The application of the drug complex as stated by claim 1, 2, 3 or 4 in the forms of prepared drug with cholagogue effect.

11. The application of the drug complex as stated by claim 1, 2, 3 or 4 in the form of prepared drug with antiphlogistic, anti-infective effect.

12. The application of the drug complex as stated by claim 1, 2, 3 or 4 in the form of prepared drug with antipyretic, analgesic effect.

13. The application of the drug complex as stated by claim 1, 2, 3 or 4 in the form of prepared drug with spasmolytic effect.

14. The application of the drug complex as stated by claim 13, with the feature that spasmolytic effect refers to inhibiting the gallbladder muscle stripe contraction.

15. The application of the drug complex as stated by claim 13, with the feature that spasmolytic effect refers to inhibiting the intestine smooth muscle contraction.

16. The identification method of drug complex soft capsule as stated by claim 3 or 4, with the feature that the method is stated as below:
Get 1 drop of soft capsule content, add 3-5 drops of H₂SO₄ and a little amount of vanillin crystal, it should be orangered, then add 1 drop of water, it should be violet;
Get 1 ml of soft capsule content, add 10 ml acetoacetate, then mix them as test solution; Additionally get mint with acetoacetate to prepare the control mint solution with 1 mg/ml concentration. According to the thin-layer chromatography experiment, aspirate 2 ul from each of the aforementioned three kind of solution respectively, drop them onto the same silica-gel G thin-layer plate, use 12-18:2-4 petroleum ether-acetoacetate at 30-60° C. as developer, develop them below 25° C., then get them out and drying them, spray with newly prepared 5% vanillin sulphate solution, blow with hot wind until the dot is clear; The chromatogram of the test solution has the same color dot in the corresponding position with the control solution chromatogram.

17. The identification method of drug complex soft capsule as stated by claim 16, with the characteristic of that the method is stated as below:
Get 1 drop of soft capsule content, add 3-5 drops of H₂SO₄ and a little amount of vanillin crystal, it should be orangered, then add 1 drop of water, it should be violet;
Get 1 ml of soft capsule content, add 10 ml acetoacetate, then mix them as test solution; Additionally get mint with acetoacetate to prepare the control mint solution with 1 mg/ml concentration; According to the thin-layer chromatography experiment, aspirate 2 ul from each of the aforementioned three kind of solution respectively, drop them onto the same silica-gel G thin-layer plate, use 15:3 petroleum ether-acetoacetate at 30-60° C. as developer, develop them below 25° C., then get them out and drying them, spray with newly prepared 5% vanillin sulphate solution, blow with hot wind until the dot is clear. The chromatogram of the test solution has the same color dot in the corresponding position with the control solution chromatogram.

18. The content measure method of drug complex soft capsule as stated by the claim 3 or 4, with the feature that the method is stated as below:
The measurement of ester content and total alcohol content Get soft capsule content about 5 g, quantify minutely, place them into the flask, add 10 ml ethanol which is indicated neutral by phenolphthalein indicator solution, mix them, and add 2 drops of phenolphthalein indicator solution. At first, neutralize the free acid with KOH solved in 0.1 mol/L ethanol, then add minutely 25 ml 0.5 mol/L KOH volumetric solution solved in ethanol, reflux for 0.5-1.5 hours in water bath, cool them, then add 0.5 ml phenolphthalein indicator solution, use 0.5 mol/l HCL to slowly titrate the residual KOH, make the blank test at the same time; 1 ml 0.5 mol/L KOH volumetric solution correspond to 99.15 mg menthol acetate(C₁₀H₂₂O₂); through the corresponding mass of the menthol acetate, the ester content of each soft capsule content should be 5-1 1% (g/g);
Acetylation: get 10 ml soft capsule content, place them into acetylating flask with 100 ml volume, add 10 ml acetic acid and 2 g newly solved anhydrous sodium acetate, with air condenser attached to the flask, placed into 145±3° C. oil bath or sand bath, boiled for 0.5-1.5 hours, get them out and cool, through condenser add 50 ml, place into water bath, vibrate, heat for 10-20 minutes, then cool them; placed into dispenser funnel, get rid of the underlayer acidic solution; add 50 ml saturated NaCl solution, vibrate fully, stationary fractionation, get rid of upper layer water solution, then wash with water for several times, 40-60 ml every time, until the washing solution is indicated neutral by phenolphthalein indicator solution; place the acquired acetylating oil into 25 ml Erlenmeyer flask with plug, add 3 g anhydrous sodium sulphate, close the flask tightly with the plug, and vibrate ever and agah, make dehydration until when getting 1 drop of acetylating oil mixed with 10 drops of CS₂ no turbidity occurs, then filtrate with dry filter paper;
Saponification: get about 1 g dry acetylating oil, quantify minutely, place into 100 ml Erlenmeyer flask, add 25 ml KOH solution solved in 0.5 mol/L ethanol, reflux for 0.5-1.5 hours in water bath, cool, take the condenser away, drop 0.5 ml phenolphthalein indicator, use 0.5 mol/L HCL solution titrate the residual KOH, make the blank test at the same time; We can get the result: Through the corresponding mass of geraniol, the total alcohol content of each soft capsule should not be less than 50.0%;
Measure the methanol and geraniol content through gas chromatography test method, chromatography condition and system adaptability experiment capillary chromatographic column: it is best to use 5% diphenyl-95% dimethyl silicane copolymer as solid capillary chromatographic column; sample inlet temperature: 200-250° C.; detector temperature: 200-250° C.; make sample measurement with the distribution ratio of 25-100:1; heat-up progressively, initial temperature 70-90° C., maintain for 2-3minutes, heat-up to 150-180° C. with the heat-up speed 4-15° C. every minute, maintain for 2-4 minutes; flow rate of carrier gas: 1-2 ml/min; According to the geraniol peak theoretical plate number should not be less than 300000;
Correction factor measurement get moderate n-pentadecane as internal standard control sample, measure precisely, add acetoacetate to prepare into 2.5 mg/ml concentration solution as internal standard solution; Get adequate amount of menthol, geraniol control sample, measure precisely, add acetoacetate to prepare into solution menthol 6 mg/ml concentration, geraniol 1 mg/ml concentration, aspirate precisely aforementioned three kinds of solution 2 ml respectively, placed into 10 ml quantifying flask, add acetoacetate, dilute it to the scale, vibrate uniformly, aspirate 1 ul, injected into gas chromatograph, measure, measure the correction factor;
Get about 50 mg soft capsule content of loading difference sample, measure precisely, placed into 10 ml quantifying flask, add 2 ml internal standard solution minutely, add acetoacetate, dilute it to the scale, vibrate uniformly, aspirate 1 ul, injected into gas chromatograph, measure, then we can acquire: according to the menthol (C₁₀H₂₀O), the herit oil in each soft capsule content should not be less than 79 mg; according to the geraniol (C₁₀H₁₈O), the essential oil should not be less than 7 mg.

19. The content measure method of drug complex soft capsule as stated by the claim No. 18, with the characteristic of that the method is stated as below: The measurement of ester content and total alcohol content: Get soft capsule content about 5 g, quantify minutely, place them into the flask, add 10 ml ethanol which is indicated neutral by phenolphthalein indicator solution, mix them, add 2 drops of phenolphthalein indicator solution. at first, neutralize the free acid with KOH solved in 0.1 mol/L ethanol, then add minutely 25 ml 0.5 mol/L KOH volumetric solution solved in ethanol, reflux for 0.5-1.5 hours in water bath, cool them, then add 0.5 ml phenolphthalein indicator solution, use 0.5 mol/l HCL to slowly titrate the residual KOH, make the blank test at the same time; 1 ml 0.5 mol/L KOH volumetric solution correspond to 99.15 mg menthol acetate(C₁₂H₂₂O₂); through the corresponding mass of the menthol acetate, the ester content of each soft capsule content should be 5-11% (g/g);
Acetylation: get 10 ml soft capsule content, place them into acetylating flask with 1 00 ml volume, add 10 ml acetic acid and 2 g newly solved anhydrous sodium acetate, with air condenser attached to the flask, placed into 145±3° C. oil bath or sand bath, boiled for 0.5-1.5 hours, get them out and cool, through condenser add 50 ml, place into water bath, vibrate, heat for 10-20 minutes, then cool them; placed into dispenser funnel, get rid of the underlayer acidic solution; add 50 ml saturated NaCl solution, vibrate fully, stationary fractionation, get rid of upper layer water solution, then wash with water for several times, 50 ml every time, until the washing solution is indicated neutral by phenolphthalein indicator solution; place the acquired acetylating oil into 25 ml Erlenmeyer flask with plug, add 3 g anhydrous sodium sulphate, close the flask tightly with the plug, and vibrate ever and agah, make dehydration until when getting 1 drop of acetylating oil mixed with 10 drops of CS₂ no turbidity occurs, then filtrate with dry filter paper;
Saponification: get about 1 g dry acetylating oil, quantify minutely, place into 100 ml Erlenmeyer flask, add 25 ml KOH solution solved in 0.5 mol/L ethanol, reflux for 0.5-1.5 hours in water bath, cool, take the condenser away, drop 0.5 ml phenolphthalein indicator, use 0.5 mol/L HCL solution titrate the residual KOH, make the blank test at the same time; We can get the result: through the corresponding mass of geraniol, the total alcohol content of each soft capsule should not be less than 50.0%;
Measure the methanol and geraniol content through gas chromatography test method, chromatography condition and system adaptability experiment, 30 m×32 mm×0.25 um capillary chromatographic column with 5% diphenyl-95% dimethyl silicane copolymer as solid capillary chromatographic column; sample inlet temperature: 240° C.; detector temperature: 240° C.; make sample measurement with the distribution ratio of 50:1; heat-up progressively, initial temperature 80° C., maintain for 2minutes, heat-up to 170° C. with the heat-up speed 8° C. every minute, maintain for 3 minutes; pneumatrophore flow rate: 1.5 ml/min; According to the geraniol peak theoretical plate number should not be less than 300000;
Correction factor measurement get moderate n-pentadecane as internal standard control sample, measure precisely, add acetoacetate to prepare into 2.5 mg/ml concentration solution as internal standard solution; Get adequate amount of menthol, geraniol control sample, measure precisely, add acetoacetate to prepare into solution menthol 6 mg/ml concentration, geraniol 1 mg/ml concentration, aspirate precisely aforementioned three kinds of solution 2 ml respectively, placed into 10 ml quantifying flask, add acetoacetate, dilute it to the scale, vibrate uniformly, aspirate 1 ul, injected into gas chromatograph, measure, measure the correction factor;
Get about 50 mg soft capsule content, measure precisely, placed into 10ml quantifying flask, add 2 ml internal standard solution minutely, add acetoacetate, dilute it to the scale, vibrate uniformly, aspirate 1 ul, injected into gas chromatograph, measure, then we can acquire: according to the menthol (C₁₀H₂₀O), the mint oil in each soft capsule content should not be less than 79 mg; according to the geraniol (C₁₀H₁₈O), the herit oil should not be less than 7 mg.

20. The quality-controlling method of drug complex soft capsule as stated by the claim No 3 or No 4, with the feature that the method is stated below:
Get 1 drop of soft capsule content, add 3-5 drops of H₂SO₄ and a little amount of vanillin crystal, it should be orangered, then add 1 drop of water, it should be violet;
Get 1 ml of soft capsule content, add 10 ml acetoacetate, then mix them as test solution; Additionally get mint with acetoacetate to prepare the control mint solution with 1 mg/ml concentration; According to the thin-layer chromatography experiment, aspirate 2 ul from each of the aforementioned three kind of solution respectively, drop them onto the same silica-gel G thin-layer plate, use 12-18:2-4 petroleum ether-acetoacetate at 30-60° C. as developer, develop them below 25° C., then get them out and drying them, spray with newly prepared 5% vanillin sulphate solution, blow with hot wind until the dot is clear. The chromatogram of the test solution has the same color dot in the corresponding position with the control solution chromatogram;
Relative density: get soft capsule content, measure the relative density according to the relative density assay method, the relative density should be 0.881˜0.901;
Refractive index: get soft capsule content, measure refractive index legally, the refractive index should be 1.456˜1.466;
Optical rotatory powder: get content as the relative density measurement, measure specific rotation legally, the specific rotation should be −14°˜−22°;
The content measurement: the measurement of ester content and total alcohol content, get soft capsule content about 5 g, quantify minutely, place them into the flask, add 10 ml ethanol which is indicated neutral by phenolphthalein indicator solution, mix them, add 2 drops of phenolphthalein indicator solution; At first, neutralize the free acid with KOH solved in 0.1 mol/L ethanol, then add minutely 25 ml 0.5 mol/L KOH volumetric solution solved in ethanol, reflux for 0.5-1.5 hours in water bath, cool them, then add 0.5 ml phenolphthalein indicator solution, use 0.5 mol/l HCL to slowly titrate the residual KOH, make the blank test at the same time; We can get the result; 1 ml 0.5 mol/L KOH volumetric solution correspond to 99.15 mg menthol acetate (C₁₂H₂₂O₂); through the corresponding mass of the menthol acetate, the ester content of each soft capsule content should be 5-11% (g/g);
Acetylation: get 10 ml soft capsule content, place them into acetylating flask with 100 ml volume, add 10 ml acetic acid and newly solved anhydrous sodium acetate, with air condenser attached to the flask, placed into 145±3° C. oil bath or sand bath, boiled for 0.5-1.5 hours, get them out and cool, through condenser add 50 ml, place into water bath, vibrate, heat for 10-20 minutes, then cool them; placed into dispenser funnel, get rid of the underlayer acidic solution; add 50 ml saturated NaCl solution, vibrate fully, stationary fractionation, get rid of upper layer water solution, then wash with water for several times, 40-60 ml every time, until the washing solution is indicated neutral by phenolphthalein indicator solution; place the acquired acetylating oil into 25 ml Erlenmeyer flask with plug, add 3 g anhydrous sodium sulphate, close the flask tightly with the plug, and vibrate ever and agah, make dehydration until when getting 1 drop of acetylating oil mixed with 10 drops of CS₂ no turbidity occurs, then filtrate with dry filter paper;
Saponification: get about 1 g dry acetylating oil, quantify minutely, place into 100 ml Erlenmeyer flask, add 25 ml KOH solution solved in 0.5 mol/L ethanol, reflux for 0.5-1.5 hours in water bath, cool, take the condenser away, drop 0.5 ml phenolphthalein indicator, use 0.5 mol/L HCL solution titrate the residual KOH, make the blank test at the same time; We can get the result, through the corresponding mass of geraniol, the total alcohol content of each soft capsule should not be less than 50.0%;
Measure the methanol and geraniol content through gas chromatography test method, chromatography condition and system adaptability experiment capillary chromatographic column: it is best to use 5% diphenyl-95% dimethyl silicane copolymer as solid capillary chromatographic column; sample inlet temperature: 200-250° C.; detector temperature: 200-250° C.; make sample measurement with the ratio of 25-100:1; heat-up progressively, initial temperature 70-90° C., maintain for 2-3minutes, heat-up to 150-180° C. with the heat-up speed 4-15° C. every minute, maintain for 2-4 minutes; flow rate of carrier gas: 1-2 ml/min; According to the geraniol peak theoretical plate number should not be less than 300000;
Correction factor measurement get moderate n-pentadecane as internal standard control sample, measure precisely, add acetoacetate to prepare into2.5 mg/ml concentration solution as internal standard solution; Get adequate amount of menthol, geraniol control sample, measure precisely, add acetoacetate to prepare into solution menthol 6 mg/ml concentration, geraniol 1 mg/ml concentration, aspirate precisely aforementioned three kinds of solution 2 ml respectively, placed into 10 ml quantifying flask, add acetoacetate, dilute it to the scale, vibrate uniformly, aspirate 1 ul, injected into gas chromatograph, measure the correction factor;
Get about 50 mg soft capsule content, measure precisely, placed into 10 ml quantifying flask, add 2 ml internal standard solution minutely, add acetoacetate, dilute it to the scale, vibrate uniformly, aspirate 1 ul, injected into gas chromatograph, measure, then we can acquire: according to the menthol (C₁₀H₂₀O), the mint oil in each soft capsule content should not be less than 79 mg; according to the geraniol (C₁₀H₁₈O), the herit oil should not be less than 7 mg.

21. The quality-control method as stated by claim No 20, with the feature the method is stated as below:
Get 1 drop of soft capsule content, add 3-5 drops of H₂SO₄ and a little amount of vanillin crystal, it should be orangered, then add 1 drop of water, it should be violet;
Get 1 ml soft capsule content, add 10 ml acetoacetate, then mix them as test solution; Additionally get mint with acetoacetate to prepare the control mint solution with 1 mg/ml concentration; According to the thin-layer chromatography experiment, aspirate 2 ul from each of the aforementioned three kind of solution respectively, drop them onto the same silica-gel G thin-layer plate, use 15:3 petroleum ether-acetoacetate at 30-60° C. as developer, develop them below 25° C., then get them out and drying them, spray with newly prepared 5% vanillin sulphate solution, blow with hot wind until the dot is clear. The chromatogram of the test solution has the same color dot in the corresponding position with the control solution chromatogram;
Relative density: get soft capsule content, measure the relative density according to the relative density assay method, the relative density should be 0.881˜0.901;
Refractive index: get soft capsule content, measure refractive index legally, the refractive index should be 1.456˜1.466;
Optical rotatory powder: get content as the relative density measurement, measure specific rotation legally, the specific rotation should be −14°−22°;
The content measurement: the measurement of ester content and total alcohol content, get soft capsule content about 5 g, quantify minutely, place them into the flask, add 10 ml ethanol which is indicated neutral by phenolphthalein indicator solution, mix them, add 2 drops of phenolphthalein indicator solution. At first, neutralize the free acid with KOH solved in 0.1 mol/L ethanol, then add minutely 25 ml 0.5 mol/L KOH volumetric solution solved in ethanol, reflux for 0.5-1.5 hours in water bath, cool them, then add 0.5 ml phenolphthalein indicator solution, use 0.5 mol/l HCL to slowly titrate the residual KOH, make the blank test at the same time; We can get the result; 1 ml 0.5 mol/L KOH volumetric solution correspond to 99.15 mg menthol acetate(C₁₂H₂₂O₂); through the corresponding mass of the menthol acetate, the ester content of each soft capsule content should be 5-11% (g/g);
Acetylation: get 10 ml soft capsule content, place them into acetylating flask with 100 ml volume, add 10 ml acetic acid and newly solved anhydrous sodium acetate, with air condenser attached to the flask, placed into 145±3° C. oil bath or sand bath, boiled for 0.5-1.5 hours, get them out and cool, through condenser add 50 ml, place into water bath, vibrate, heat for 10-20 minutes, then cool them; placed into dispenser funnel, get rid of the under layer acidic solution; add 50 ml saturated NaCl solution, vibrate fully, stationary fractionation, get rid of upper layer water solution, then wash with water for several times, 50 ml every time, until the washing solution is indicated neutral by phenolphthalein indicator solution; place the acquired acetylating oil into 25 ml Erlenmeyer flask with plug, add 3 g anhydrous sodium sulphate, close the flask tightly with the plug, and vibrate ever and agah, make dehydration until when getting 1 drop of acetylating oil mixed with 10 drops of CS₂ no turbidity occurs, then filtrate with dry filter paper;
Saponification: get about 1 g dry acetylating oil, quantify minutely, place into 100 ml Erlenmeyer flask, add 25 ml KOH solution solved in 0.5 mol/L ethanol, reflux for 0.5-1.5 hours in water bath, cool, take the condenser away, drop 0.5 ml phenolphthalein indicator, use 0.5 mol/L HCL solution titrate the residual KOH, make the blank test at the same time; We can get the result, through the corresponding mass of geraniol; the total alcohol content of each soft capsule should not be less than 50.0%;
Measure the methanol and geraniol content through gas chromatography test method, chromatography condition and system adaptability experiment, 30 m×32 mm×0.25 um capillary chromatographic column with 5% diphenyl-95% dimethyl silicane copolymer as solid capillary chromatographic column; sample inlet temperature: 240° C.; detector temperature: 240° C.; make sample measurement with the distribution ratio of 50:1; heat-up progressively, initial temperature 80° C., maintain for 2minutes, heat-up to 170° C. with the heat-up speed 8° C. every minute, maintain for 3 minutes; pneumatrophore flow rate: 1.5 ml/min; According to the geraniol peak theoretical plate number should not be less than 300000;
Correction factor measurement get moderate n-pentadecane as internal standard control sample, measure precisely, add acetoacetate to prepare into2.5 mg/ml concentration solution as internal standard solution; Get adequate amount of menthol, geraniol control sample, measure precisely, add acetoacetate to prepare into solution menthol 6 mg/ml concentration, geraniol 1 mg/ml concentration, aspirate precisely aforementioned three kinds of solution 2 ml respectively, placed into 10 ml quantifying flask, add acetoacetate, dilute it to the scale, vibrate uniformly, aspirate 1 ul, injected into gas chromatograph, measure the correction factor;
Get about 50 mg soft capsule content, measure precisely, placed into 10 ml quantifying flask, add 2 ml internal standard solution minutely, add acetoacetate, dilute it to the scale, vibrate uniformly, aspirate 1 ul, injected into gas chromatograph, measure, then we can acquire: according to the menthol (C₁₀H₂₀O), the mint oil in each soft capsule content should not be less than 79 mg; according to the geraniol (C₁₀H₁₈O), the herit oil should not be less than 7 mg.

22. The method as stated by claim No 18, 19, 20 or 21, with the characteristic of that n-pentadecane in the method can be substituted by n-tridecane or n-tetradecane or naphtalene or camphor or n-undecane.
